(19) United States
(12) Patent Application Publication
(10) Pub. No.: US 2023/0144070 A1
(43) Pub. Date: May 11, 2023

(54) CONNECTOR OF AN IMPLANTABLE MEDICAL DEVICE

(71) Applicant: BIOTRONIK SE & Co. KG, Berlin (DE)

(72) Inventors: Ringo MANNHAUPT, Luckenwalde (DE); Martin BOHMEYER, Neuenhagen (DE)

(73) Assignee: BIOTRONIK SE & Co. KG, Berlin (DE)

(21) Appl. No.: 17/911,940

(22) PCT Filed: Apr. 12, 2021
(86) PCT No.: PCT/EP2021/059441

§ 371 Date: Sep. 15, 2022

(30) Foreign Application Priority Data
Apr. 21, 2020 (EP) 20170571.2

(51) Int. Cl. H01R 12/57

(52) U.S. Cl. CPC H01R 12/57; H01R 2201/12

(57) ABSTRACT

A connector of an implantable medical device is connectable to a mating connector of an assembly to be connected to the implantable medical device along an insertion direction. The connector comprises a contacting device for making electrical contact with an electrical contact element of the mating connector, wherein the contacting device comprises a housing and a contact spring arranged in the housing. The contact spring forms a multiplicity of head sections for electrically contacting the electrical contact element of the mating connector, a multiplicity of first foot sections electrically contacting the housing and a multiplicity of second foot sections electrically contacting the housing, the first foot sections being arranged at a first axial position and the second foot sections at a second axial position different than the first axial position, when viewed along the insertion direction.

### CROSS-REFERENCE TO RELATED APPLICATIONS

This application is the United States National Phase under 35 U.S.C. § 371 of PCT International Patent Application No. PCT/EP2021/059441, filed on Apr. 12, 2021, which claims the benefit of European Patent Application No. 20170571.2, filed on Apr. 21, 2020, the disclosures of which are hereby incorporated by reference herein in their entireties.

### TECHNICAL FIELD

The present invention concerns a connector of an implantable medical device according to the preamble of claim 1.

### BACKGROUND

A connector of this kind is connectable to a mating connector of an assembly to be connected to the implantable medical device along an insertion direction. The connector comprises a contacting device for making electrical contact with an electrical contact element of the mating connector, wherein the contacting device comprises a housing and a contact spring arranged in the housing.

An implantable medical device may, for example, be formed by a pulse generator of a stimulation system, for example, a pacemaker or defibrillation system or a neuro-stimulation system. Such a pulse generator can, for example, be implanted subcutaneously into a patient, wherein electrodes are usually connectable to the pulse generator for implantation at a location of interest, for example, in the heart of a patient, in order to cause stimulation at the location of interest.

For connecting an assembly, for example, an electrode, to an implantable medical device, for example, a pulse generator, a connector is used which can be mated with an associated mating connector to establish an electrical connection between the assembly and the implantable medical device. Generally, high demands for such medical connectors for use on medical devices exist. Connectors should be biocompatible. In addition, connectors must be able to provide for a good electrical connection with high conductivity and low, essentially invariable electrical resistance. The connection of an assembly, for example, an electrode, to an implantable medical device should be simple and convenient, with a reliable and safe connection during operation.

Conventional connectors for use on implantable medical devices, such as known from U.S. Publication No. 2003/0157846 A1 or U.S. Publication No. 2005/0234521 A1, usually have a contacting device, in which a contact spring in the form of an annular helical spring is enclosed in a housing and is elastically deformable in such a way that a mating connector in the form of a contact pin can be inserted into an insertion opening formed by the housing and surrounded circumferentially by the contact spring in order to make contact with the housing via the contact spring. In U.S. Publication No. 2005/0234521 A1, herein, coil turns of the contact spring are arranged at a skewed angle, which allows the coil turns of the contact spring to slide in a scraping fashion along the mating connector when the mating connector is mated to the connector, in this way scraping off any oxide film potentially present on the mating connector to improve an electrical contact between the mating connector and the connector. Such contact springs are also referred to as canted coil springs.

To provide a biocompatible connector, a contact spring of a contacting device is made, for example, of a platinum or iridium material. Platinum or iridium usually has a low elasticity, which makes it necessary to form the contact spring such that it may be deformed within a range of elasticity, while allowing a repeated connection of a connector to a corresponding mating connector with a low risk of failure of the contact spring, especially when taking into account the usual sizes for medical connectors in the millimeter range.

The present disclosure is directed toward overcoming one or more of the above-mentioned problems, though not necessarily limited to embodiments that do.

### SUMMARY

It is an objective of the instant invention to provide a connector of an implantable medical device that may provide for a reliable connection to an associated mating connector.

At least this objective is achieved by a connector comprising the features of claim 1.

Accordingly, the contacting device forms a multiplicity of head sections for electrically contacting the electric contact element of the mating connector, a multiplicity of first foot sections electrically contacting the housing and a multiplicity of second foot sections electrically contacting the housing, the first foot sections being arranged at a first axial position and the second foot sections at a second axial position different than the first axial position, when viewed along the insertion direction.

The contact spring forms head sections for electrically contacting the electrical contact element of the mating connector. If the connector is mated with the mating connector along the insertion direction, the contact element of the mating connector comes into contact with the contact spring of the contacting device, such that an electrical contact in between the contact element of the mating connector and the contact spring is established. By means of its foot sections, herein, the contact spring is in abutment and hence in electrical contact with the housing of the contacting device, such that via the contact spring an electrical connection in between the contact element of the mating connector and the housing of the connector is established, once the connector is mated with the mating connector.

The shape of the contact spring herein diverts from the shape of a regular helically wound spring. In particular, the contact spring forms different foot sections, first foot sections being arranged at a first axial position and second foot sections being arranged at a second axial position, with reference to the insertion direction. The first foot sections and the second foot sections hence are axially displaced with respect to each other, such that a contacting abutment in between the contact spring and the housing of the contacting device is established via different foot sections at axially different positions.

The foot sections hence are placed at axially different positions. The foot sections herein, in one embodiment, may be joined with each other via the head sections, such that the contact spring may form an opening in between the first foot sections and the second foot sections, the contact spring extending within a surface of revolution, the surface having a U-shape or a horseshoe-shape in space and being rotated about a center axis to form the contact spring.

In one embodiment, the contact spring has a meandering shape. The contact spring may, for example, be formed by a continuous wire or may be cut from surface element, such as a planar plate element or a hollow tubing, the foot sections and the head sections being formed by adjoining, meandering sections of the contact spring.

In one embodiment, the contact spring extends along a direction of extension, wherein the head sections are aligned along the direction of extension. The direction of extension may, for example, extend longitudinally, the contact spring hence forming a longitudinal, straight element, particularly after forming of the contact spring but before assembly of contact spring and housing. In other words, a straight formed contact spring has to be formed in to an annular form before assembly with the housing. Alternatively, the direction of extension may be curved, for example, about a center axis, the contact spring hence forming a ring element which may be a circumferentially closed or may be circumferentially opened in that ends of the contact spring are not fixedly connected to each other.

In one embodiment, the head sections are aligned with each other along the direction of extension. The head sections hence are spaced with respect to each other along the direction of extension, wherein the head sections may be spaced at equal distances or at unequal distances.

In one embodiment, the first foot sections and the second foot sections alternate, when viewed along the direction of extension, a first foot section in each case being followed by a second foot section and vice versa. The first foot sections and the second foot sections thus are interleaved, when viewed along the direction of extension, a first foot section at its first axial position being followed by a second foot section at the second axial position, such that the foot sections are staggered along the direction of extension and in addition are axially displaced with respect to each other along the insertion direction.

In one embodiment, the head sections are curved about the direction of extension. Each head section herein may connect a first foot section at the first axial position to a second foot section at the second axial position, such that the first foot sections and the second foot sections are linked via the head sections, the head sections hence bridging the first foot sections at the first axial position on a first side of the contact spring and the second foot sections at the second axial position on a second side of the contact spring. Due to their curved shape, the head sections may come into abutment with a contact element of a mating connector once the mating connector is connected with the connector, such that an electrical contact in between the contact element of the mating connector and the contact spring and via the contact string to the housing may be established.

In one embodiment, the contact spring extends about the insertion opening, such that a contact element of a mating connector may be received radially within the contact spring for contacting with the head sections of the contact spring for establishing an electrical connection in between the mating connector and the contacting device of the connector.

Herein, the head sections beneficially face towards the center axis, such that the head sections may abut and hence electrically contact with the contact element of the mating connector once the mating connector is inserted into the insertion opening of the contacting device.

In one embodiment, each head section extends within an associated skewed plane which forms a skewed angle to the direction of extension. The head section may be curved within the skewed plane, wherein due to the orientation of the skewed plane the head section is arranged at a skewed angle with respect to the direction of extension of the contact spring and hence, if the contact spring extends circumferentially about a center axis and is formed by a surface of revolution, to a radial direction with respect to the center axis. Due to the skewed orientation of the head sections, the head sections may be tilted and thus radially moved under elastic tensioning of the contact spring when the mating connector is plugged into the connector, such that, when the connector is mated with the mating connector, the head sections are elastically tensioned by elastic deformation of the contact spring and thus bear with elastic tension against the contact element of the mating connector when the connector and the mating connector are mated with each other.

The skewed orientation of the head sections thus provides a defined elasticity at the contact spring. The inclined position of the head sections in addition allows achieving a beneficial contact between the head sections and the contact element of the mating connector.

In one embodiment, each of the first foot sections and/or each of the second foot sections are curved about an associated rotational axis which extends substantially along the insertion direction. The foot sections hence comprise a curvature, the curvature providing for a turn in a plane substantially perpendicular to the insertion direction. The foot sections herein may provide for a deformation zone in which the contact spring is elastically deformable when mating the connector with a corresponding mating connector, the foot sections hence defining a rotational axis for the head sections about which neighboring head sections may be tilted with respect to each other. Particularly, each foot section may act as a counter-bearing or common rotation axis for two neighboring head sections, particularly such that both head sections move substantially parallel when the mating connector is plugged into the connector.

Particularly, the whole contact spring, i.e., each part of contact spring including head and foot section, is configured to be deformed under elastic tensioning of the contact spring when the mating connector is plugged into the connector.

In one embodiment, each first foot section is joined via a first leg to one of the head sections and via a second leg to another of the head sections. The first foot sections hence are linked to adjoining head sections via legs, wherein the legs may, in one embodiment, be arranged such that they are not parallel with respect to each other. The legs may be arranged with respect to each other such that they extend from the associated foot section and are arranged at a skewed angle with respect to one another so that they approach one another (taper) towards the adjoining head sections.

The first leg and the second leg, for example, may be arranged in a common first plane which extends substantially perpendicular to the insertion direction. The first plane herein may be placed at the first axial position such that the legs are arranged at the same axial position as the first foot sections.

Alternatively or in addition, in one embodiment, each second foot section is joined via a third leg to one of the head sections and via a fourth leg to another of the head sections.

The second foot sections hence are linked to adjoining head sections via legs, wherein the legs may, in one embodiment, be arranged such that they are not parallel with respect to each other. The legs may be arranged with respect to each other such that they extend from the associated foot section and are arranged at a skewed angle with respect to one another so that they approach one another (taper) towards the adjoining head sections.

The third leg and the fourth leg, for example, may be arranged in a common second plane which extends substantially perpendicular to the insertion direction. The second plane herein may be placed at the second axial position such that the legs are arranged at the same axial position as the second foot sections.

The housing can be used, for example, to establish an electrical connection to an electrical assembly of the implantable medical device, for example, by connecting an electrical line to the housing.

A connector of the type described may be located on an implantable medical device in the form of a pulse generator of a stimulation system, for example, a pacemaker or defibrillation system or a neuro-stimulation system. Such a pulse generator can, for example, be implanted subcutaneously in a patient, wherein an electrical assembly can be connected to a pulse generator, for example, an electrode, which is to be implanted at a location of interest, for example, in the heart of a patient, in order to cause a stimulation at the location of interest.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The idea underlying the present invention shall subsequently be explained in more detail with reference to the embodiments shown in the figures. Herein:

FIG. 1 shows a view of the human heart with an implanted stimulation system;

FIG. 2 shows a schematic view of a terminal block of a pulse generator of the stimulation system;

FIG. 3 shows a perspective view of an embodiment of a contacting device of a connector for electrically contacting with a contact element of a mating connector;

FIG. 4 shows a front view of the contacting device;

FIG. 5 shows a perspective view of a contact spring of the contacting device;

FIG. 6 shows a front view of the contact spring;

FIG. 7 shows a side view of the contact spring;

FIG. 8 shows a partially cut view of the contacting device;

FIG. 9A shows a front view of the contacting device, with a mating connector mated with the contacting device;

FIG. 9B shows a sectional view along line A-A as shown in FIG. 9A;

FIG. 10 shows a view of a part for forming the contact spring, in an intermediate step during manufacturing;

FIG. 11 shows a view of the part of FIG. 10, after bending foot sections relative to head sections;

FIG. 12 shows a bottom view of the party according to FIG. 11;

FIG. 13 shows a side view of the part according to FIG. 11;

FIG. 14 shows a front view of the part according to FIG. 11;

FIG. 15 shows a view of a part for forming the contact spring, in another embodiment; and

FIG. 16 shows a front view of the part according to FIG. 15.

### DETAILED DESCRIPTION

In the following, embodiments of the instant invention are described with reference to the figures. In the figures, components having like functions are denoted using the same reference signs.

It shall be noted that the embodiments described herein are not to be understood as restrictive for the present invention, but merely serve the purpose of illustration.

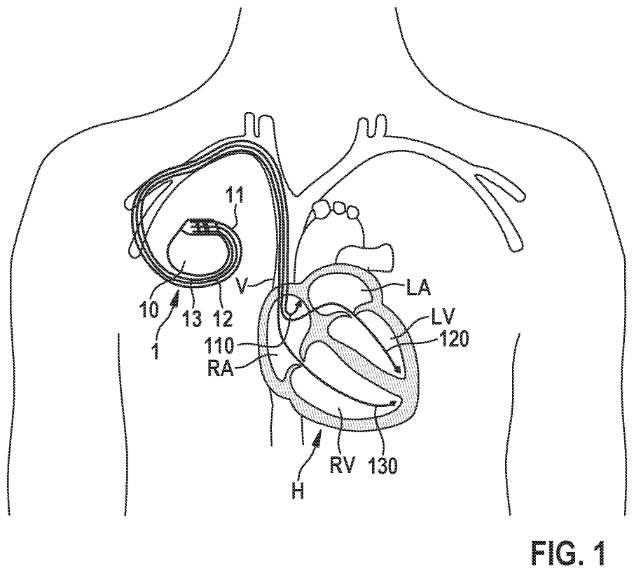

FIG. 1 shows a schematic view of the heart H of a patient with an implanted stimulation system, for example, in the form of a so-called CRT system. The stimulation system 1 comprises a pulse generator 10 to which electrodes 11, 12, 13 are connected. The pulse generator 10 is implanted together with the electrodes 11, 12, 13 in the patient in such a way that the electrodes 11, 12, 13 extend from the pulse generator 10 through the upper vena cava V to the heart H and to different stimulation locations in the heart H.

In the example shown, three electrodes 11, 12, 13 are connected to the pulse generator 10. The pulse generator 10, for example, is implanted subcutaneously in the area of the patient's collarbone. From the pulse generator 10 the electrodes 11, 12, 13 extend in such a way that the electrodes 11, 12, 13 come to rest with their distal ends 110, 120, 130, for example, in the right atrium RA (electrode 11 with the distal end 110), in the left ventricle LV (electrode 12 with the distal end 120) and in the right ventricle RV (electrode 13 with the distal end 130) and thus a stimulation may take place at different stimulation locations in the heart H via stimulation pulses generated by the pulse generator 10 and fed to the electrodes 11, 12, 13.

In cardiac resynchronization therapy, for example, stimulation takes place in the left is ventricle LV and in the right ventricle RV, so that stimulation can bring about synchronicity of ventricular activity. In this way, the pumping capacity of the heart H is to be increased in patients with chronic cardiac insufficiency.

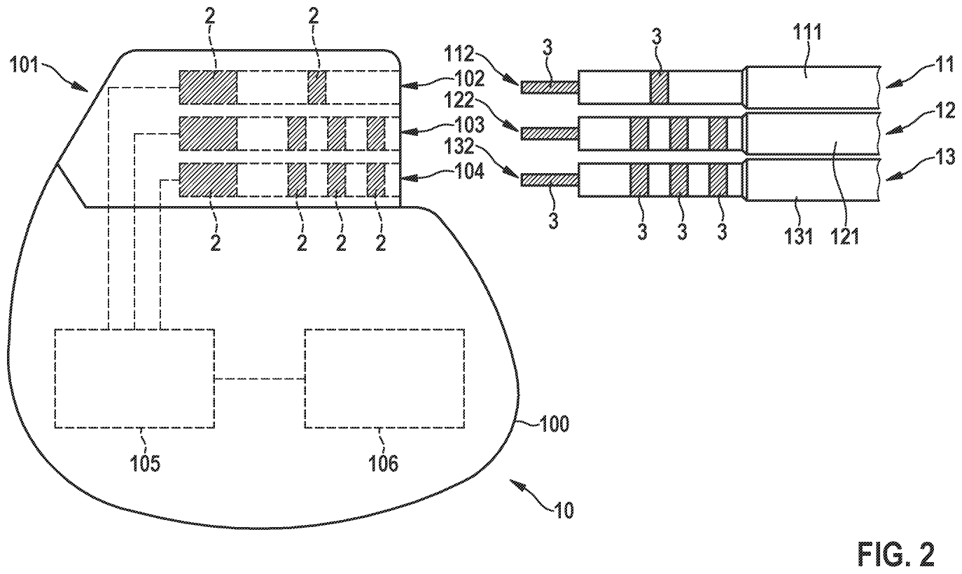

As schematically shown in an embodiment in FIG. 2, the pulse generator 10 has a housing 100 in which electrical and electronic components in the form of a control device 105 and a power supply device 106 in the form of a battery are enclosed and encapsulated.

A terminal block 101 is arranged on the housing 100 and has connectors 102, 103, 104 in the form of socket connectors, into which electrodes 11, 12, 13 with associated mating connectors 112, 122, 132 in the form of plugs can be inserted in order to establish an electrical connection between the electrodes 11, 12, 13 and the connectors 102, 103, 104 and thus the pulse generator 10.

The connectors 102, 103, 104, for example, are standardized and are designed as IS-1 or IS4 connections according to ISO 27186:2010.

The connectors 102, 103, 104 each have electrical contacting devices 2 for electrically contacting with contact elements 3 of mating connectors 112, 122, 132, wherein a first connector 102, for example, may have two contacting devices 2 and the other two connectors 103, 104 each may have four contacting devices 2. The connector 102 is thus designed as a two-pole connection, while the other connectors 103, 104 are designed as four-pole connections.

Depending on the design of the connectors 102, 103, 104, the mating connectors 112, 122, 132 of electrodes 11, 12, 13 are designed as IS-1 plugs (mating connector 112) or IS4 plugs (mating connectors 122, 132), for example, and have two electrical contact elements 3 (two-pole mating connector 112) or four electrical contact elements 3 (four-pole mating connectors 122, 132).

The contacting devices 2 of the connectors 102, 103, 104 and the contact elements 3 of the mating connectors 112, 122, 132 are designed in such a way that a pair-wise electrical contact is established between the respectively associated contacting devices 2 and contact elements 3 when the respective connector 102, 103, 104 and mating connector 112, 122, 132 are mated with each other. Each contacting device 2 thus serves to establish an electrical connection with an associated contact element 3 of a mating connector 112, 122, 132 when the mating connector 112, 122, 132 is mated with an associated connector 102, 103, 104.

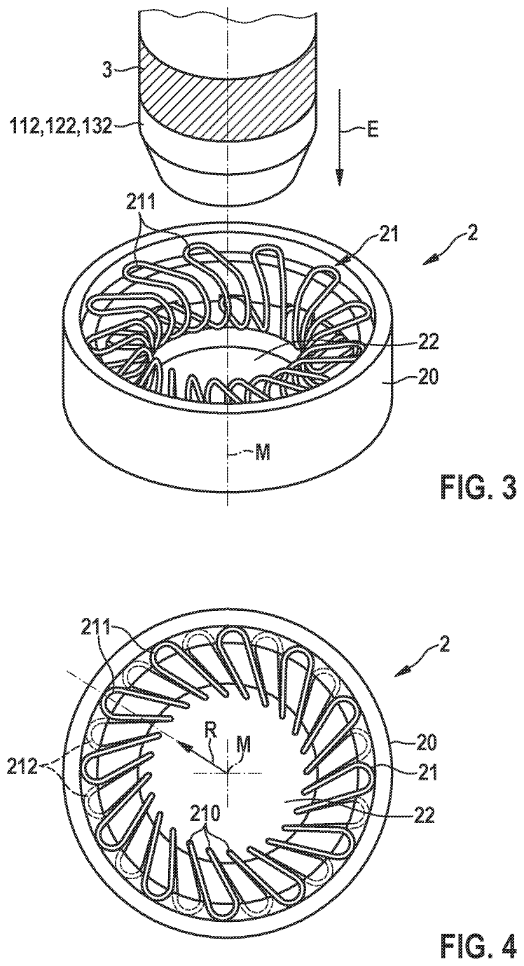

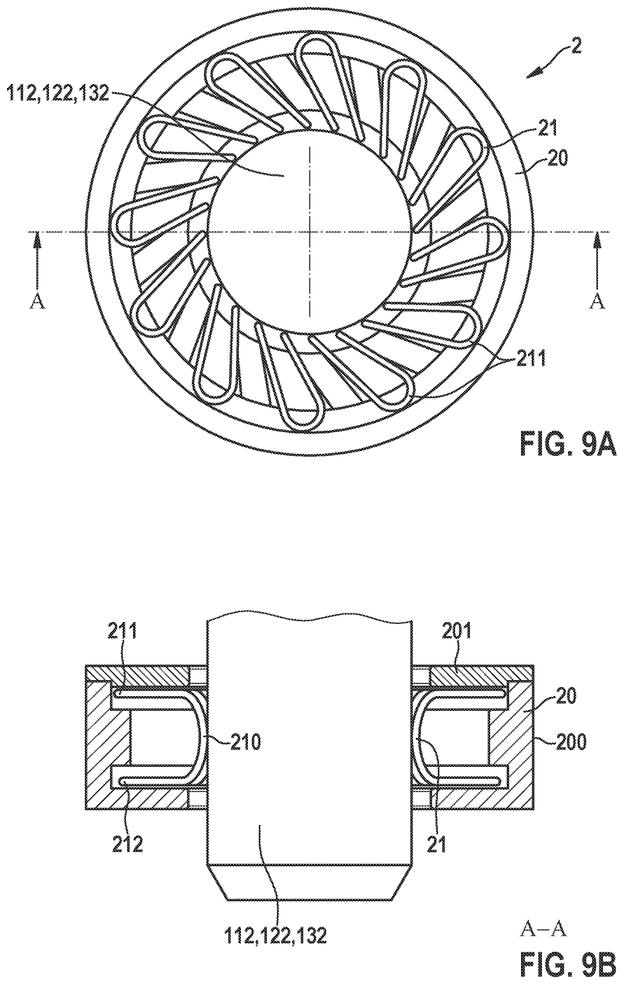

FIGS. 3 to 9A, 9B show an embodiment of a contacting device 2 which has a substantially annular shape and forms an insertion opening 22 into which an associated mating connector 112, 122, 132 can be inserted along an insertion direction E, which is aligned with a center axis M concentric with the contacting device 2, so that a contact element 3 arranged on the mating connector 112, 122, 132 comes to lie radially inside the contacting device 2.

In the shown embodiment, the contacting device 2 comprises a housing 20 which receives a contact spring 21 for contacting with a contact element 3 of a mating connector 112, 122, 132. The shape of the contact spring 21 diverts from the shape of a regular, helically wound spring in that it forms head sections 210 facing towards the center axing M of the contacting device 2 and foot sections 211, 212 placed radially outside of the head sections 210, the foot sections 211, 212 being axially displaced along the center axis M with respect to one another.

While the head sections 210 facing radially inwards serve to contact with a contact element 3 of a mating connector 112, 122, 132 inserted along the insertion direction E into the contacting device 2, as illustrated in FIG. 3, the contact spring 21 by means of the foot sections 211 abuts with the housing 20 and hence is in electrical contact with the housing 20.

As visible from FIG. 3 in view of FIG. 8, first foot sections 211 are arranged at a first axial side of a ring section 204 protruding radially inwards inside the housing 20, and second foot sections 212 are arranged at a second side of the ring section 204. The foot sections 211, 212 hence are axially displaced, wherein each foot section 211, 212 is in abutment with the housing 20 and hence electrically contacts the housing 20.

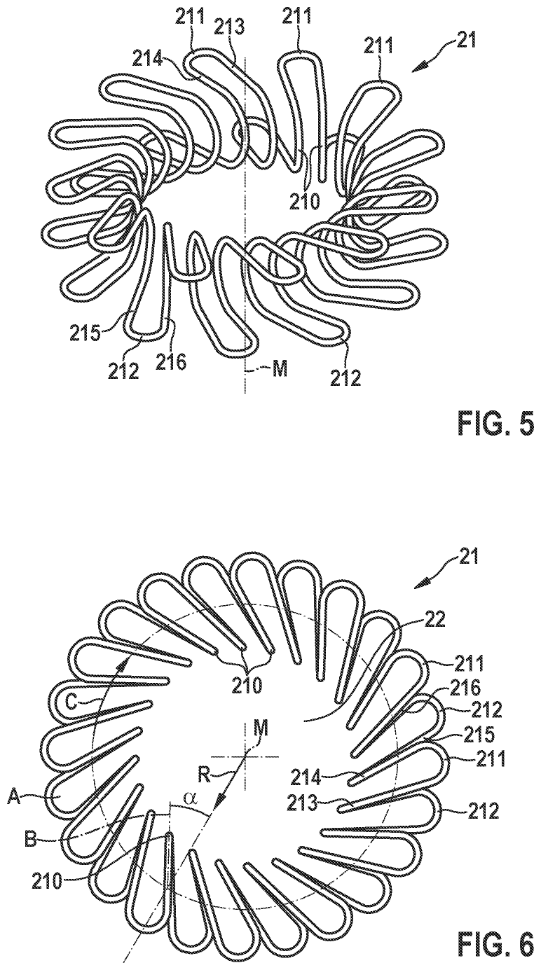

As visible from FIG. 3 in view of, for example, FIG. 5 and FIG. 8, the contact spring 2 extends along a surface of revolution, the surface of revolution being formed by a U shape or a horseshoe shape which is revolved in space about the center axis M. In particular, the contact spring 21 extends along a surface which is opened at a side facing radially outwards.

Referring now to FIG. 6, the contact spring 21 extends along a direction of extension C, the direction of extension C being curved about the center axis M along a circle about the center axis M. The head sections 210 are aligned along the direction of extension C, wherein the head sections 210 are equally spaced with respect to each other and face radially inwards.

As visible from FIG. 6 in view of FIG. 5, the head sections 210 are curved about the direction of extension C, such that the head sections 210 have a rounded shape.

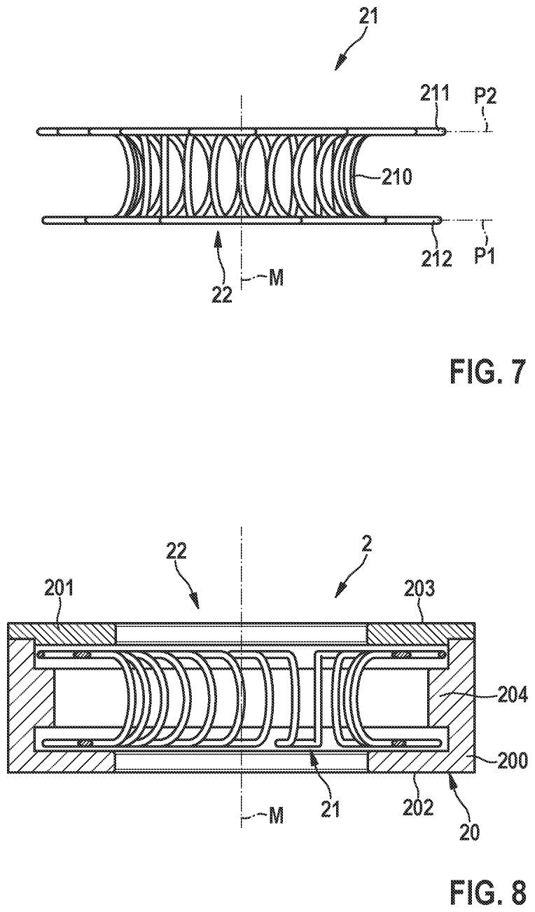

Each head section 210 herein links a first foot section 211 at a first axial side of the contact spring 21 to a second foot section 212 at a second axial side of the contact spring 21. Each foot section 211, 212 is curved about an associated axis of rotation A, the first foot sections 211 being arranged in a first plane P1 at a first axial position, as illustrated in FIG. 7, and the second foot sections 212 being arranged within a second plane P2 at a second axial position. The first foot sections 211 and the second foot sections 212 hence are axially displaced with respect to each other.

Because each foot section 211, 212 is curved about an associated axis of rotation A, it provides for a turn within the associated plane P1, P2. Each first foot section 211 herein, as visible from FIG. 5, via an associated pair of legs 213, 214 is linked to adjoining head sections 210. Each second foot section 212 is joined to adjoining head sections 210 via an associated pair of legs 215, 216.

As visible from FIG. 6, the legs 213, 214, 215, 216 of each pair of legs adjoining a foot section 211, 1212 do not extend in parallel to one another, but taper towards the head sections 210.

As visible from FIG. 5 in view of FIG. 6, the first foot sections 211 and the second foot sections 212, in addition to being axially displaced along the center axis M, are staggered along the direction of extension C, such that each first foot section 211 at the first axial position is followed by a second foot section 212 at the second axial position, as in particular visible from FIG. 6.

In addition, as visible from FIG. 6, the head sections 210 each extend within a skewed plane B being arranged at a skewed angle α with respect to the direction of extension C and a radial direction R. Due to the skewed arrangement of the head sections 210 and the legs 213, 214, 215, 216 adjoining the head sections 210 at either axial side of the contact spring 21, the head sections 210 may be elastically moved radially outwards when inserting a mating connector 112, 122, 132 into the insertion opening 22 formed by the contacting device 2, such that the contact spring 21 is elastically deformed by bending the head sections 210 and the adjoining legs 213, 214, 215, 216 and the head sections 210 come to rest under elastic pretension on the contact element 3 of the mating connector 112, 122, 132.

Due to the rounded shape of the head sections 210, the head sections 210 may easily slide onto the contact elements 3 of the mating connector 112, 122, 132, when inserting the mating connector 112, 122, 132 in the insertion direction E into the insertion opening 22 of the contacting device 2. Due to the skewed arrangement of the head sections 210 and due to the elastic pretensioning forces caused by deformation of the contact spring 21 when inserting the mating connector 112, 122, 132, the head sections 210 may scrape through an oxide layer potentially present on the contact element 3, such that an electrical contact with the contact element 3 is improved.

Referring now to FIG. 8, the housing 20 is formed by a housing part 200 which is fixedly connected to a housing cover 201. Herein, the housing part 200 forms, on a circumferential outer wall, the ring section 204 facing radially inwards.

For fabricating the contacting device 2, the contact spring 21 is arranged within the housing part 200 prior to fixing the housing cover 201 to the housing part 200, wherein after insertion of the contact spring 21 into the housing part 200 the housing cover 201 is arranged on the housing part 200 and is fixed to connected to the housing part 200, for example, by gluing or welding.

The contact spring 21 may be formed from a continuous wire, which is bent to assume a meandering shape, as a visible, for example, from FIG. 5. The continuous wire herein may be circumferentially closed in that ends of the wire are connected to each other, wherein alternatively the continuous wire may be circumferentially opened in that the ends of the wire are not connected to each other.

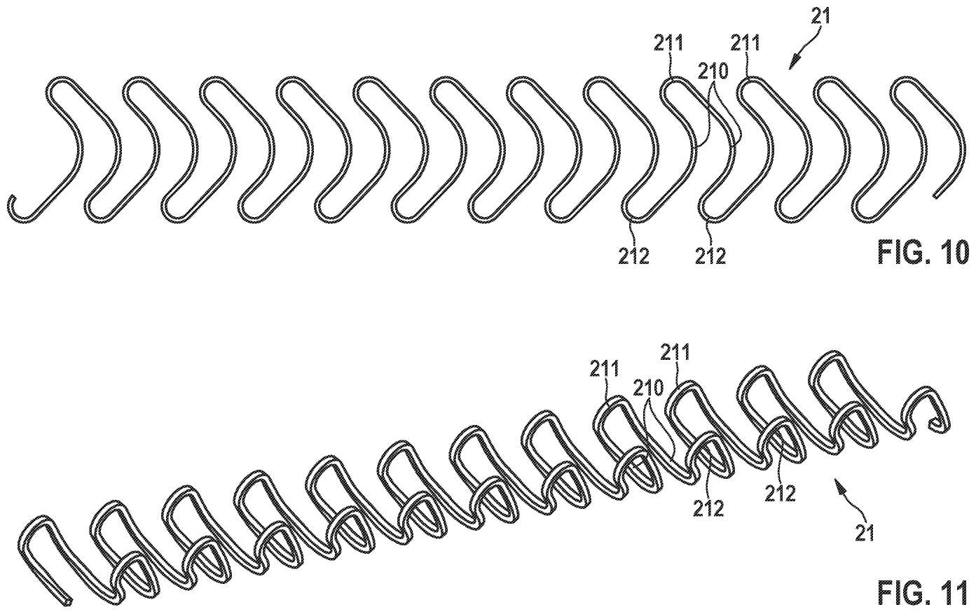

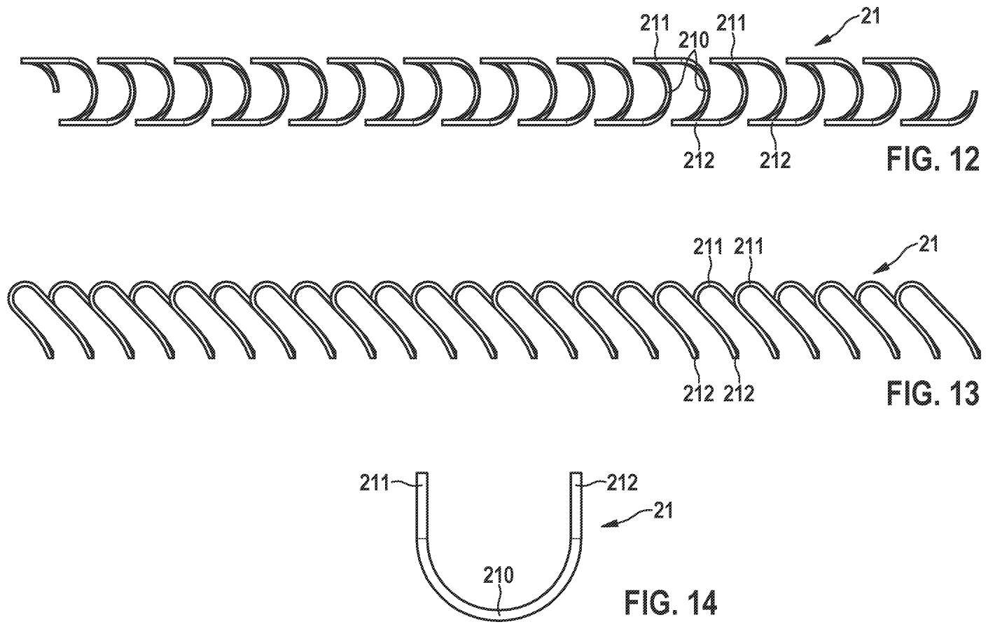

In an alternative embodiment, as shown in FIG. 10, a part forming the contact spring 21 may be cut, for example, using laser cutting, from a plate-like element, such that in an intermediate state during fabrication a planar element is formed as shown in FIG. 10. By bending the foot sections 211, 212 with respect to the head sections 210, as shown in FIGS. 11 to 14, the contact spring 21 is formed, which prior to inserting it into the housing 20 extends straight along a longitudinal direction.

For placing the contact spring 21 on the housing 20, the contact spring 21 may be bent such that the contact spring 21 assumes an annular shape, as visible from FIGS. 3 to 9A, 9B, wherein prior to inserting the contact spring 21 into the housing 20 ends of the contact spring 21 may be fixedly connected to each other, or alternatively the ends may be left open such that the contact spring 21 is circumferentially opened.

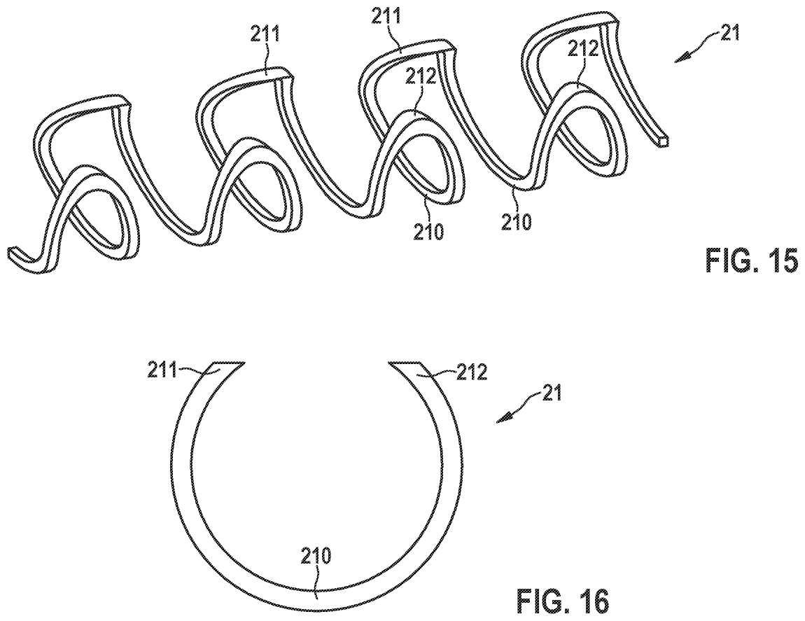

In yet another embodiment, shown in FIGS. 15 and 16, the contact spring 21 may be cut, for example, using laser cutting, from a hollow tube element, such that after cutting the contact spring 21 extends straight along a longitudinal direction, as visible from FIG. 15. In this embodiment, legs linking the foot sections 211, 212 to the head sections 210 are curved, corresponding to the curvature of the hollow tube element from which the contact spring 21 is cut.

In either case, the contact spring 21 may, for example, be made from a platinum or iridium material to obtain a biocompatible connector 102, 103, 104.

The idea underlying the present invention is not limited to the embodiments described above, but may be implemented in entirely different ways.

A connector of the type described herein can be used on an implantable medical device that is, for example, part of a stimulation system or another system. In general, a connector can be used, e.g., on a therapeutic system or on a diagnostic system, for example, a sensor system or a recording system, wherein such a connector can be used to provide for a connection for an electrode or another (electrical) assembly.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 Stimulation system
10 Implantable medical device (pulse generator)
100 Housing
101 Terminal block
102-104 Connector
105 Controller
106 Energy supply device
11 Sub-assembly (electrode)
110 Distal end
111 Proximal end
112 Mating connector
12 Sub-assembly (electrode)
120 Distal end
121 Proximal end
122 Mating connector
13 Sub-assembly (electrode)
130 Distal end
131 Proximal end
132 Mating connector
2 Contacting device
20 Housing
200 Housing part
201 Housing cover
202, 203 Side wall
204 Ring section
21 Contact spring
210 Head sections
211, 212 Foot sections
213-216 Legs
22 Insertion opening
3 Electrical contact element
α Skewed angle
A Rotational axis
B Skewed plane
C Direction of extension
E Insertion direction
H Heart
LA Left atrium
LV Left ventricle
M Center axis
P1, P2 Plane
R Radial direction
RA Right atrium
RV Right ventricle
V Upper vena cava

1. Connector of an implantable medical device, the connector being connectable to a mating connector of an assembly to be connected to the implantable medical device along an insertion direction, the connector comprising a contacting device for making electrical contact with an electrical contact element of the mating connector, wherein the contacting device comprises a housing and a contact spring arranged in the housing, wherein the contact spring forms a multiplicity of head sections for electrically contacting the electrical contact element the mating connector, a multiplicity of first foot sections electrically contacting the housing and a multiplicity of second foot sections electrically contacting the housing, the first foot sections being arranged at a first axial position and the second foot sections at a second axial position different than the first axial position, when viewed along the insertion direction.

2. Connector according to claim 1, wherein the contact spring has a meandering shape.

3. Connector according to claim 1, wherein the contact spring extends along a direction of extension, wherein the head sections are aligned along the direction of extension.

4. Connector according to claim 3, wherein the first foot sections and the second foot sections alternate, when viewed along the direction of extension, a first foot section each case being followed by a second foot section vice versa.

5. Connector according to claim 3, wherein the head sections are curved about the direction of extension.

6. Connector according to claim 3, wherein the direction of extension is directed about a center axis of the contacting device, wherein the contacting device forms an insertion opening receiving the mating connector.

7. Connector according to claim 6, wherein the head sections face towards the center axis.

8. Connector according to claim 3, wherein each head section extends within an associated skewed plane which forms a skewed angle to the direction of extension.

9. Connector according to claim 1, wherein each of the first foot sections and/or each of the second foot sections are curved about an associated rotational axis which extends substantially along the insertion direction.

10. Connector according to claim 1, wherein each first foot section is joined via a first leg to one of said head sections and via a second leg to another of said head sections.

11. Connector according to claim 10, wherein the first leg and the second leg are arranged non-parallel to one another.

12. Connector according to claim 10, wherein the first leg and the second leg are arranged in a common first plane which extends substantially perpendicularly to the insertion direction.

13. Connector according to claim 1, wherein each second foot section is joined via a third leg to one of said head sections and via a fourth leg another of said head sections.

14. Connector according to claim 13, wherein the third leg and the fourth leg are arranged non-parallel to one another.

15. Connector according to claim 13, wherein the third leg and the fourth leg are arranged in a common second plane which extends substantially perpendicularly to the insertion direction.
